(19) United States
(12) Patent Application Publication
(10) Pub. No.: US 2022/0244729 A1
(43) Pub. Date: Aug. 4, 2022

(54) BABY TRANSPORT AND METHOD FOR OPERATING THE SAME

(71) Applicants: Yu-Sian Jiang, Austin, TX (US); Mu-Jen Huang, Taipei (TW)

(72) Inventors: Yu-Sian Jiang, Austin, TX (US); Mu-Jen Huang, Taipei (TW)

(21) Appl. No.: 17/164,901

(22) Filed: Feb. 2, 2021

(51) Int. Cl. G05D 1/02; B62B 9/00; A61B 5/00

(52) U.S. Cl. CPC G05D 1/0212; G05D 1/0088; A61B 5/6887; B62B 9/00

(57) ABSTRACT

A baby transport is provided. The baby transport includes a car body, a first sensing unit, a second sensing unit, and a processing unit. The car body is configured to carry a baby. The first sensing unit is configured to sense a biological signal of the baby. The second sensing unit is configured to sense an environment context. The processing unit is configured to determine a target of interest according to the biological signal of the baby and the environment context; plan a route according to the environment context and the target of interest; and control the car body to move according to the route.

### FIELD

The present disclosure generally relates to a baby transport, and a method for operating the same.

### BACKGROUND

A general way for moving a conventional baby transport such as a baby stroller requires a caregiver to push or pull the stroller with a handle. A baby walker, on the other hand, could be unsafe when obstacles are present. Therefore, it is desirable to provide an autonomous baby transport for safety and convenience.

### SUMMARY

In one aspect of the present disclosure, a baby transport is provided. A baby transport includes a car body, a first sensing unit, a second sensing unit, and a processing unit. The car body is configured to carry a baby. The first sensing unit, coupled to the car body, is configured to sense a biological signal of the baby. The second sensing unit, coupled to the car body, is configured to sense an environment context. The processing unit, coupled to the first sensing unit and the second sensing unit, is configured to determine a target of interest according to the biological signal of the baby and the environmental context; plan a route according to the environment context and the target of interest; and control the car body to move according to the route.

In another aspect of the present disclosure, a method of operating a baby transport is provided. The method includes the following actions. A biological signal of the baby is sensed by a first sensing unit. An environmental context is sensed by a second sensing unit. A target of interest is determined by the processing unit according to the biological signal of the baby and the environmental context. A route is planned by the processing unit according to the environment context and the target of interest. And, a baby car body is controlled, by the processing unit, to move according to the route.

### BRIEF DESCRIPTION OF THE DRAWINGS

FIG. 1 is a block diagram of a baby transport according to an implementation of the present disclosure.

FIG. 2 is a schematic diagram of a baby transport according to an implementation of the present disclosure.

FIG. 3 is a flowchart of a method for operating a baby transport according to an embodiment of the present disclosure.

FIG. 4 is a schematic diagram illustrating the determination of the target of interest according to an embodiment of the present disclosure.

FIG. 5 is a schematic diagram of a cost map.

FIG. 6 is a flowchart of a method for operating a baby transport according to another embodiment of the present disclosure.

FIG. 7 is a schematic diagram illustrating the route planning and the re-planning according to yet another embodiment of the present disclosure.

FIGS. 8A-8B are schematic diagrams illustrating the determination of a biological status of the baby according to the biological signals.

### DETAILED DESCRIPTION

The following description contains specific information pertaining to exemplary implementations in the present disclosure. The drawings in the present disclosure and their accompanying detailed description are directed to merely exemplary implementations. However, the present disclosure is not limited to merely these exemplary implementations. Other variations and implementations of the present disclosure will occur to those skilled in the art. Unless noted otherwise, like or corresponding elements among the figures may be indicated by like or corresponding reference numerals. Moreover, the drawings and illustrations in the present disclosure are generally not to scale, and are not intended to correspond to actual relative dimensions.

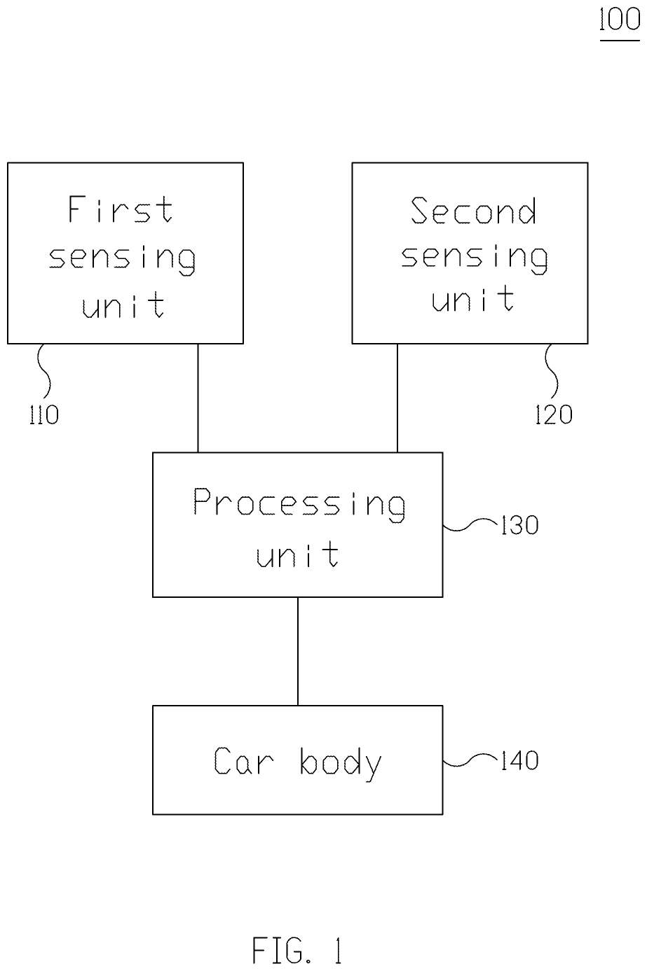

FIG. 1 is a block diagram of a baby transport 100 according to an implementation of the present disclosure. The baby transport 100 includes a first sensing unit 110, a second sensing unit 120, a processing unit 130, and a car body 140. The car body 140 is configured to carry a baby. The first sensing unit 110, coupled to the car body 140, is configured to sense a biological signal of the baby. For instance, the biological signal may include, but not limited to, an image, a sound, a voice, a speech, a heart rate, a breath, or the combination of the above.

In one implementation, the first sensing unit 110 may include an image capturing unit (e.g., camera) for capturing the images of the baby. The first sensing unit 110 may be a depth-sensing camera with a depth sensor, an RGB camera, or an infrared (IR) camera. In some embodiments, the first sensing unit 110 may include a light source (e.g., an IR illuminator or a visible light illuminator) for lighting the environment. The camera may be a camera module further includes an image processing unit such as high dynamic range (HDR) imaging for improving the image quality, or a format conversion such as serdes. The image may be processed by a processing unit to understand the baby status including emotion, sleeping, vigilance, comfortableness, health, or activity from either a static image frame or a video consists of image stream. In another implementation, the first sensing unit 110 further includes a voice recording unit configured to record a voice or sound of the baby. In some implementations, the first sensing unit 110 further includes a thermal sensor configured to sense the body temperature of the baby. In some other implementations, the first sensing unit 110 further includes a heartbeat rate (HBR) monitor configured to detect the HBR of the baby. In some other implementations, the first sensing unit 110 further includes a breath monitor. The HBR monitor or the breath monitor may be realized by an image capturing device to detect the HBR via image recognition.

The second sensing unit 120, coupled to the car body 140, is configured to sense an environment context. For example, the environment context may include, but not limited to, an image of the environment, or information about the distance to an object/obstacle, a location, a position, or a movement of an object/obstacle. In one implementation, the second sensing unit 120 may include an image capturing unit for capturing images of the environment, such as, a photo sensor, a depth-sensing camera with a depth sensor, an RGB color camera, or an infrared (IR) camera. In some embodiments, the second sensing unit 120 further includes a light source (e.g., an IR illuminator or a visible light illuminator) for illuminating the environment. In another implementation, the second sensing unit 120 may include a lidar sensor, a radar, or an ultrasonic sensor, for detecting object(s)/obstacle(s) and provide information about the object(s)/obstacle(s). In some implementations, the second sensing unit 120 may include a Global Positioning Satellite (GPS) receiver and/or an inertial measurement unit (IMU) for global/local positioning and obtaining trajectory of the vehicle, externally applied forces, vehicle movement, and road dynamics. In some implementations, the second sensing unit 120 may include an accelerometer for detecting bumping or for positioning.

The processing unit 130 is coupled to the car body 140, the first sensing unit 110, and the second sensing unit 120. The processing unit 130 may receive data, process data, and generate instructions for the baby transport. In one embodiment, the processing unit 130 may be a hardware module comprising one or more central processing unit (CPU), microcontroller(s), ASIC, or a combination of above but is not limited thereof. The processing unit 130 may perform computer vision technique, such as object detection and recognition, and/or image processing. In one embodiment, the processing unit 130 is configured to perform a biometric detection/recognition according to the images captured by the first sensing unit 110. The biometric detection/recognition may include face detection, facial recognition, head pose detection, eyes openness detection, yawning detection, gaze detection, body skeleton detection, gender detection, age detection, or a combination of above, but is not limited thereof. In some other embodiments, the processing unit 130 may further determine a biological status including drowsy, sleep, microsleep, vigilance, emotion, comfortableness, intrigued, hungry, etc., based on the biometric detection/recognition and the biological signal sensed by the first sensing unit 110. Furthermore, the processing unit 130 may detect the breathing, the heartbeat rate of the baby, and/or perform other biological recognitions via computer vision technique to obtain the biological status of the baby and determine whether the baby is breathing normally. In addition, the processing unit 130 may monitor the movement of the baby and track the activity of the baby. In some embodiments, the processing unit 130 may further perform a voice recognition based on the voice or sound of the baby recorded by the first sensing unit 110, for example, a crying or a yelling. In an embodiment, the processing unit 130 may detect and process the environment context sensed from the second sensing unit 120 and provide the representation of the environment dynamic. In one embodiment, the processing unit 130 may process the images captured by the second sensing unit 120, and perform object detection. In another embodiment, the processing unit 130 may process the sensed data of a lidar, a radar, or an ultrasonic sensor, and perform an obstacle detection. In yet another embodiment, the processing unit 130 may track the object and the obstacle by a fusion of multiple sensor data. In one embodiment, the processing unit 130 may perform a localization, determine an orientation of the baby transport, and build a map according to the sensed data from a camera, GPS, IMU, and/or an encoder for motor angular and velocity. In another embodiment, the processing unit 130 may create a point cloud and a cost map according to the sensed data from the second sensing unit. In some embodiments, the processing unit 130 performs path/route planning and controls the motion of the baby transport.

In some other embodiments, the baby transport 100 further includes an escalation unit configured to give a sound or voice, and/or send an alarm or notification to a mobile device via a wireless transceiver. The wireless transceiver may be a WIFI, mobile 4G/5G module, BLE, but is not limited thereof, for data uplink and downlink communications.

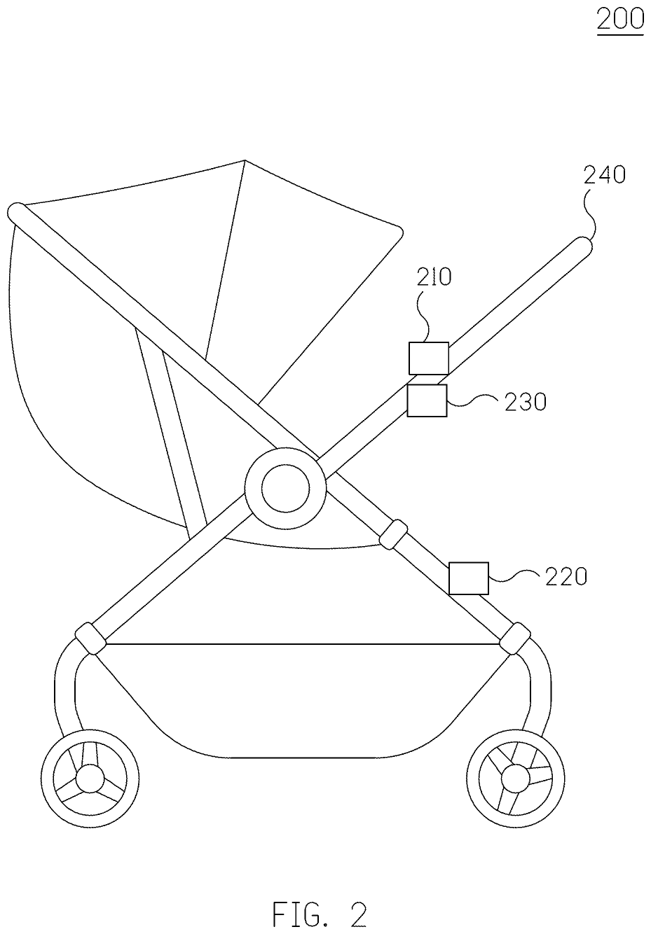

FIG. 2 is a schematic diagram of a baby transport 200 according to an implementation of the present disclosure. As shown in FIG. 2, the baby transport 200 includes a car body 240. For instance, the car body includes a seat for carrying the baby, wheels, and/or a handle. In one embodiment, the first sensing unit 210 configured to sense a biological signal of the baby is disposed around the seat of the car body 240, and the second sensing unit 220 configured to sense an environment context is disposed on the car body 240 at a position with sight clearance. The processing unit 230 is disposed around the seat of the car body 240. In another embodiment, the second sensing unit 220 is disposed near the handle of the car body 240. However, the mechanical structures of the baby transport may vary depending on the design and application, and the arrangement of the sensing units 210 and 220 may differ.

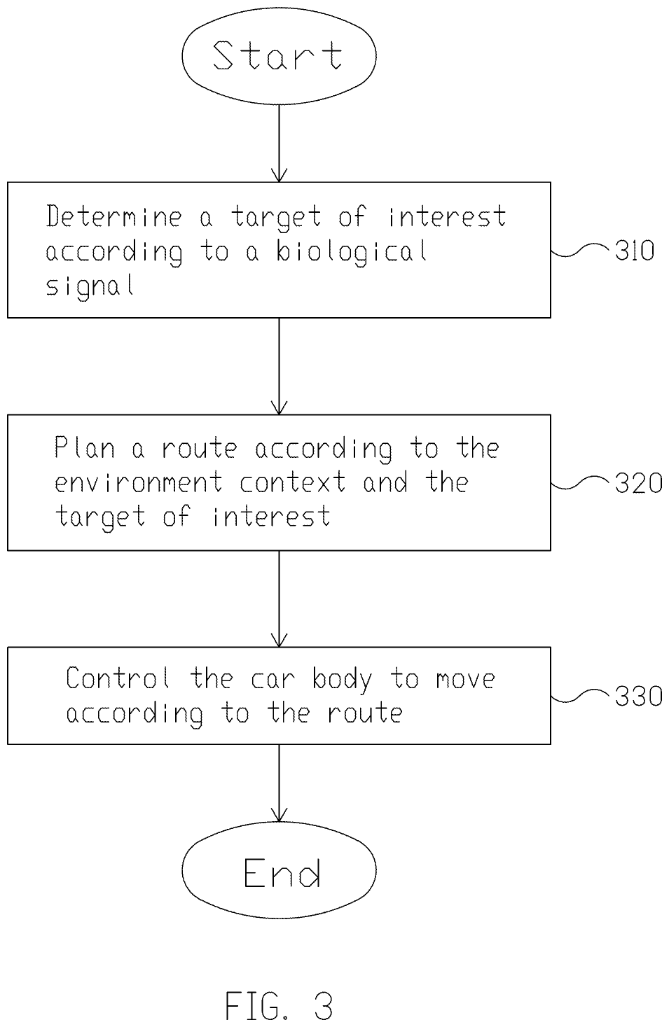

FIG. 3 is a flowchart of a method for operating a baby transport according to an embodiment of the present disclosure. The method includes the following actions. In action 310, the processing unit determines a target of interest according to a biological signal and the environment context. The biological signal of the baby, sensed by the first sensing unit, may include, but not limited to, an image, a sound, a voice, a speech, a heart rate, a breath, or the combination of the above. The target of interest may include, but not limited to an object, a person, a location, a direction, or an area. For example, the processing unit determines whether the baby is excited or intrigued about an object, a person, a location, or a direction, according to the biological signal and the environment context. Specifically, the processing unit perform computer vision technique on the captured images to obtain a head pose, a head movement, a facial expression, a facial feature, a facial gesture, a body pose, an eyeball movement, an eye openness status, an eyes blink velocity and amplitude and a ratio between velocity and amplitude, an eyes gaze vector, a gaze point, a body skeleton and its movement, or a gesture, and then determine whether the baby is looking at, facing at, or interacting with an object, a person, a location, a direction, or an area; and thus determine the corresponding object, person, location, direction, or area as the target of interest.

In another implementation, the target of interest is further determined according to a biological status of the baby. The processing unit determines the biological status of the baby by performing biometric detection/recognition. The biological status may include, but not limited to, drowsy, sleep, microsleep, emotion, comfortableness, hungry, intrigued, or a body language. Each biological status corresponds to an object, a person, a location, a direction, or an area. For instance, when a drowsy status is identified, the processing unit may infer the bedroom as the target of interest.

In some implementations, the first sensing unit further includes a microphone adapted to record the sound or voice of the baby. The processing unit may perform sound recognition, voice recognition and/or speech recognition to determine the target of interest. For example, when the baby calls his/her mom or dad, the processing unit determines the baby's target of interest is his/her mom or dad. Additionally, a baby's voice or sound may be recorded in advance to represent an object, a location, a direction, or a person, and thus the target of interest of the baby could be identified by sound recognition, voice recognition and/or speech recognition. Moreover, the processing unit may determine the target of interest further according to other types of biological signal such as a heartbeat rate or a breath of the baby. For example, when a drowsy or sleep status is inferred according to a heartbeat rate, the bedroom may be inferred as the target of interest.

In action 320, the processing unit plans a route according to the environment context and the target of interest inferred from action 310. For example, the processing unit may determine the coordinate of the target of interest according to a map established according to the environment context and a position of the baby transport, then plan a route accordingly. When the target of interest is an object, a person, or an area, the processing unit sets a destination near the object, the person, or the area, and plans the route to the destination coordinate accordingly. When the target of interest is a direction, the processing unit may set a destination by a specific distance along the direction. Meanwhile, the processing unit further process the environment context sensed by the second sensing unit and provide a representation of static or dynamic environment that includes a map, a cost map, an obstacle position and its shape, an orientation/heading of the baby transport, an object, a recognized object, an object ID, a point cloud, a context primitive, a road quality, a friction of the road surface, or a slope of a tilt road. The map may be a global map or a local map constructed by SLAM (Simultaneous localization and mapping). The cost map is built from the sensed data for characterizing the cost of traveling through the environment. The obstacle position and shape may be obtained from a 3D camera, lidar, a radar, or an ultrasonic sensor. Each object may be given an object ID, and the object recognized by the image recognition may be correlated to the object ID. A point cloud including sensed data points in space may be constructed by the obstacle information to establish a relationship between perception from different sensors, and may be used for tracking the obstacle dynamic in an image stream of the environment, where each obstacle being tracked may be given an object ID. The context primitive is a pre-defined scenario or state that represents a combination of the environment context dynamic. For example, a man is drinking water is a pre-defined context primitive. The context primitive may also be a combination or relation of environment objects that characterizes a scenario, for instance, a congestion level, or an environment safety evaluation. According to the environment context and the target of interest, the processing unit plans the route to navigate the baby transport to the target of interest without colliding with obstacles.

In action 330, the processing unit controls the car body to move according to the route. For instance, the processing unit may include a motor controller to generate a control command for steering the baby transport to move to a waypoint according to the planned route. The control command may include a steering angle, an angular velocity, a throttle command and/or a brake. As a result, the baby transport infers a baby's target of interest to navigate the baby to the target autonomously and safely according to the environment context, and thus relieves the burden of the caregiver.

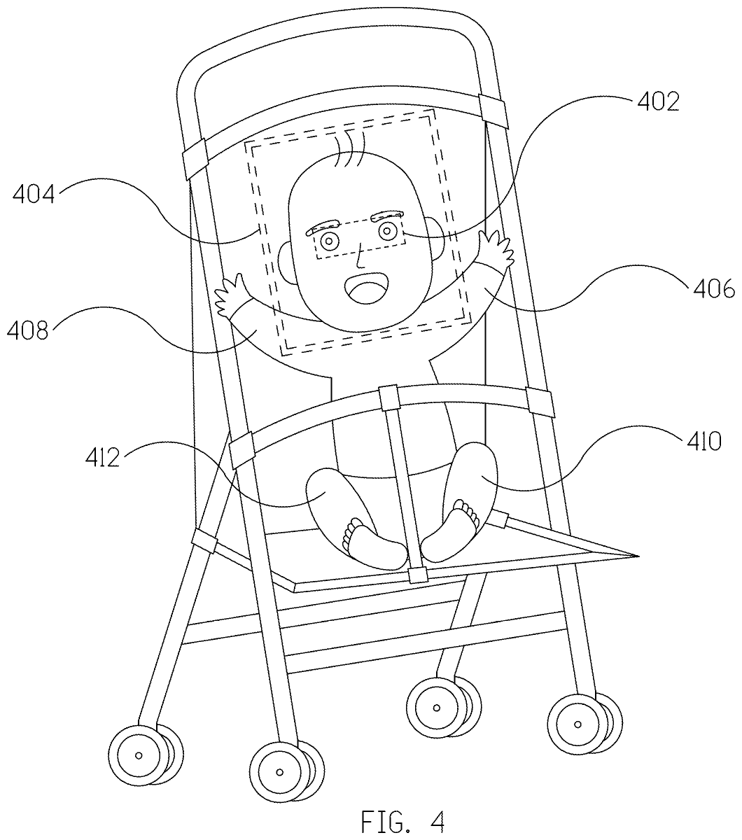

FIG. 4 is a schematic diagram illustrating the determination of the target of interest according to an embodiment of the present disclosure. As stated above, the processing unit perform computer vision technique on the captured images. As shown in FIG. 4, the processing unit may identify the head 404, eyes/gaze 402, hands 406, 408, legs 410, 412 and/or other biological feature of the baby, and determine the target of interest accordingly. For instance, when the baby is intrigued to look at the left, the processing unit may determine the baby's target of interest is an object, a person, or a location on the left side. Similarly, when the baby turning his or her head, or points his or her arms or legs toward a specific direction, the processing unit determines the specific direction as the baby's target of interest. In another embodiment of present invention, the processing unit may determine a target of interest according to a time threshold of gaze fixation. For example, when a baby is staring at a certain point for a period of time (e.g., 3 seconds), the point is identified as the target of interest.

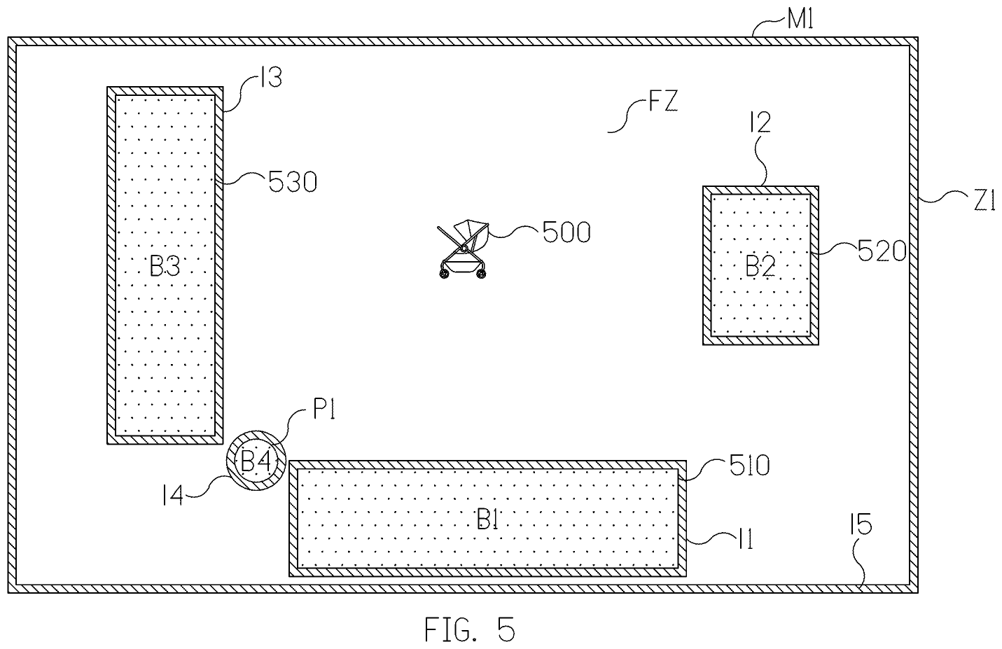

FIG. 5 illustrates a cost map Ml. The cost map includes a baby transport 500 in a living room Z1, a sofa 510, a desk 520, a cabinet 530, and a person P1. The cost map assigns a number of values to each grid of the map for characterizing the cost in terms of the obstacle distance, where the forbidden region(s) that overlaps with the obstacles (e.g., dotted area B1, B2, B3, B4) may be assigned to a highest value, e.g., 255, while the inflation area (e.g., diagonal line area I1, I2. I3, I4, I5) serving as a guard band to a safety region may be assigned to a secondary high value, e.g., 250, and the rest of the grid (e.g., FZ) may be assigned to a value characterized from 0 to 250 according to the distance from the obstacle, or a constant value from 0 to 250. As a result, a path planner may determine a route by finding a minimum aggregated cost value of all waypoints, and thus to compromise between the obstacle proximity and the route distance. Thereafter, the processing unit may control and actuate the motor to navigate the baby transport to the target of interest without colliding with any obstacle. During the navigation, the processing unit may track the obstacles continuously and update the cost map in real-time, in order to update the route according to obstacle dynamic.

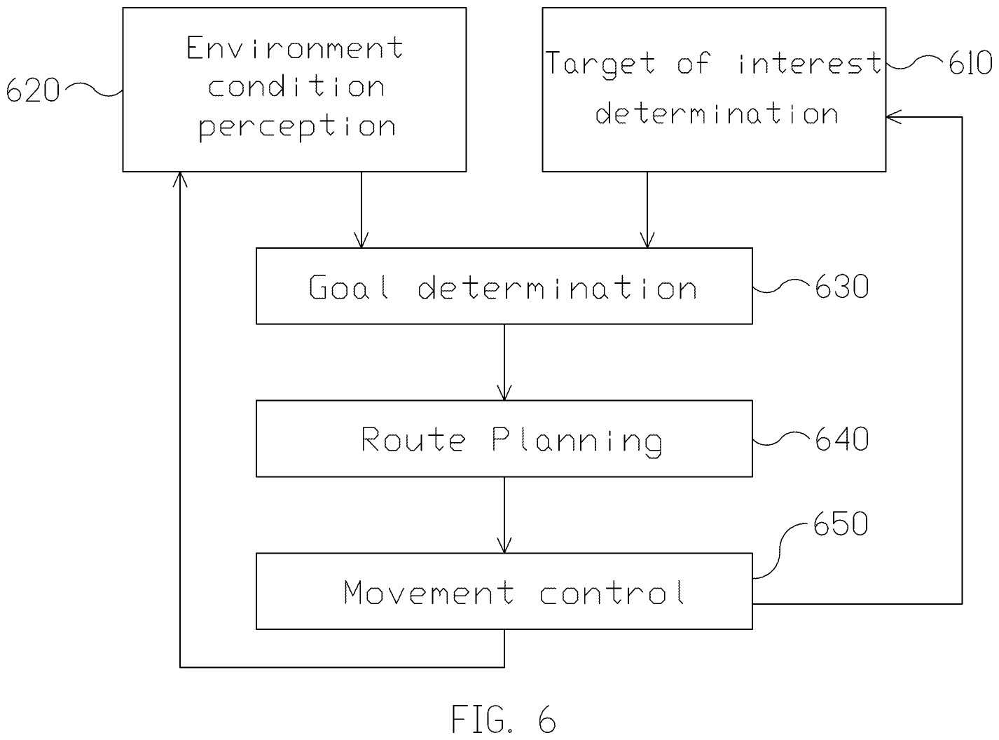

FIG. 6 is a flowchart of a method for operating a baby transport according to another embodiment of the present disclosure. In this embodiment, a goal is modified according to a context primitive. The context primitive is a pre-defined scenario or state that represents a combination of the environment context dynamic. The method includes the following actions. In action 610, the processing unit determines a target of interest according to the biological signal, the biological status, and/or the environment context. In action 620, the environment condition is perceived. In action 630, a goal is derived according to the target of interest and the environment condition, e.g., a map. For instance, the goal is a destination coordinate of the target of interest in a map. In action 640, the processing unit plans a route from current position of the baby transport to the goal, where the route consists of a plurality of waypoints. In action 650, the processing unit controls and actuates the motor to move according to the waypoints. However, in some implementations, the waypoint may be recognized to be unsafe for the baby transport according to the context primitive. In such a case, the route may be modified to avoid passing through the unsafe waypoint. In another case, when the goal is recognized to be unsafe for the baby transport, the processing unit may modify the goal to a secondary option of the target of interest, or stop the baby transport when there's no safe alternative.

In another embodiment, a reward may be used for justifying and modifying the inferred target of interest. Firstly, the processing unit determines an inferred target according to the biological signal, the biological status, and/or the environment context. Secondly, the processing unit may plan the route and actuate a motor to move the baby transport to the target of interest. When the vehicle is moving, the environment context will be changed dynamically, and thus a baby may react on the change of the environment context. As a result, the reaction of the baby may be monitored and evaluated as a reward to justify the level of confidence for the inferred target of interest. The reaction of the baby may be, for example, a feedback of his/her emotion status, where the emotion may be characterized from his/her face appearance or facial expression, voice, breath rate, and/or HBR, etc. For instance, when the emotion is rated as excited or positive, the reward is given by a higher value such that the baby transport remains the route of the navigation, or accelerate/decelerate the baby transport. On the other hand, if the baby's emotion is rated as fear or negative, the reward is given a lower value such that the baby transport may implement a maneuver, such as, stop moving, decelerate, clear, and avoid this target of interest, make a turn to change the heading of the car body, and/or re-evaluate a new target of interest.

In one embodiment when the baby transport has reached its target of interest, or there's not a target of interest been inferred at a given time point, the baby transport may stop and result in no movement. For instance, when the baby is detected as sleep, the baby transport may stop or move to a designated place. In another embodiment, the baby transport may further tilt down the seat of the car body when the biological status of the baby is detected as sleep.

In another embodiment when there is no new target of interest inferred, the baby transport may remain on the route to the last target of interest.

In yet another embodiment when a new target of interest is inferred while the baby transport has not reached the former target of interest, the baby transport may determine a new goal and re-plan a route according to the new target of interest. For example, when the baby transport is on the way to a first goal according to a first target of interest, the processing unit may re-evaluate a second goal according to the second target of interest.

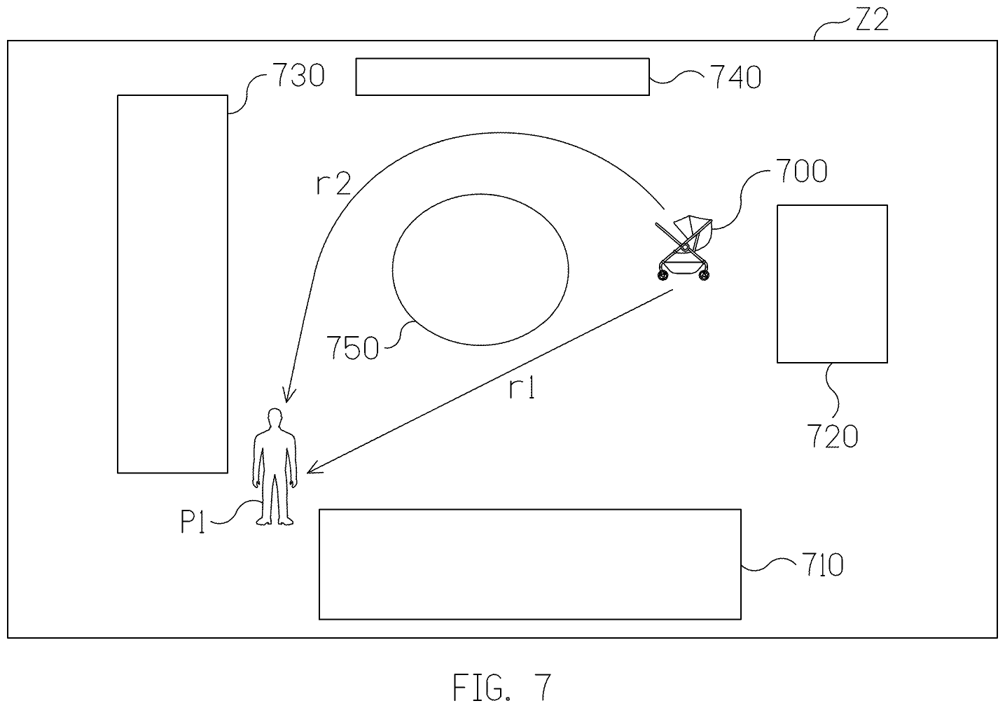

FIG. 7 is a schematic diagram illustrating the route planning and the re-planning according to yet another embodiment of the present disclosure. A baby transport 700 is in a living room Z2, and the environment has obstacles including a sofa 710, a desk 720, a cabinet 730, a TV 740 and a person P1. When a target of interest is determined as the person P1, the baby transport may set the goal to the coordinate near the person P1, and navigate to the goal via the route r1. On the other hand, when the TV 740 is inferred as a target of interest, the goal is therefore determined as a coordinate near the TV 740. However, in the midway, when a second target of interest, said the person P1 is inferred, the system may update the goal to a coordinate of near the person P1, and update the route as r2.

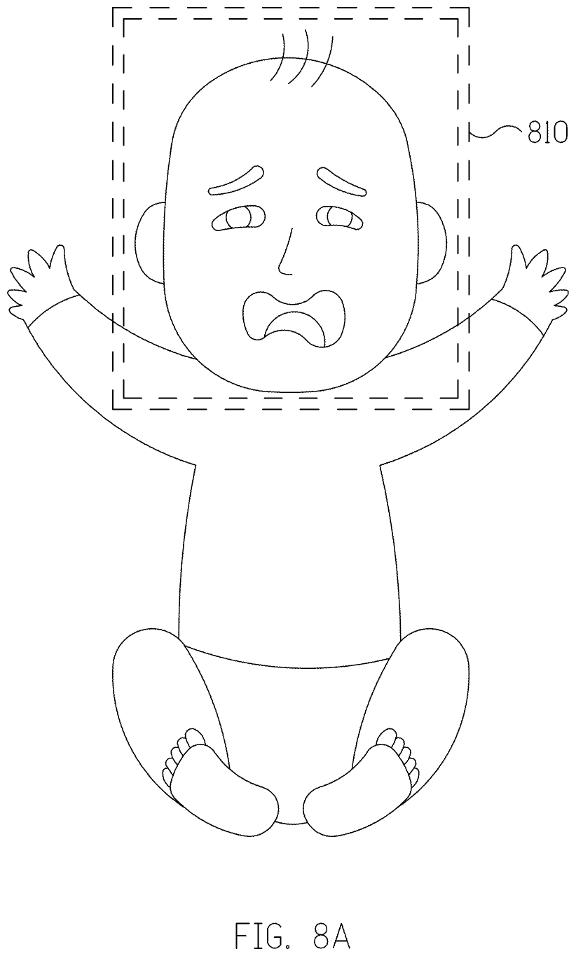

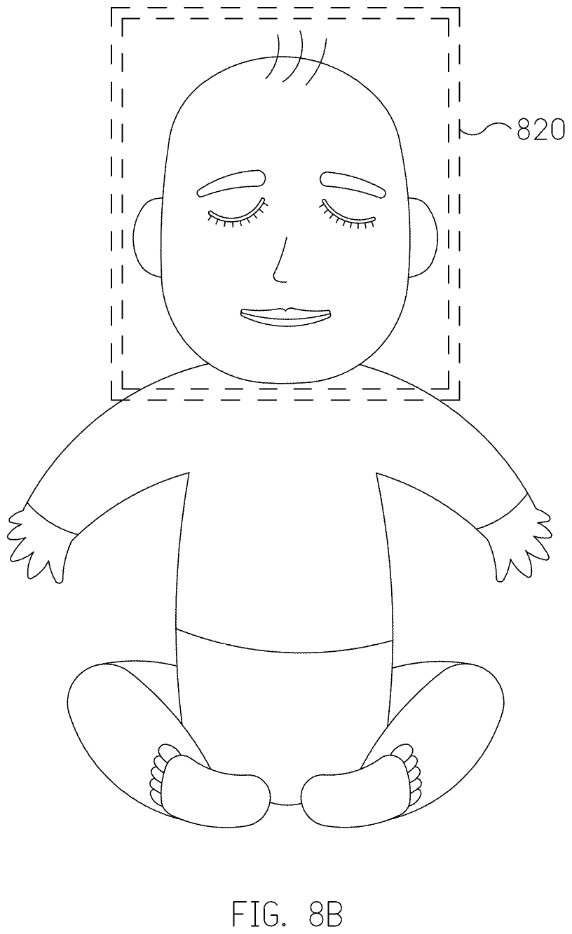

FIGS. 8A-8B are schematic diagrams illustrating the determination of a biological status of the baby according to the biological signals. Specifically, a biological status of the baby is determined according to the biological signal of the baby. As shown in FIG. 8A, the processing unit may perform computer vision technique on the captured images to obtain a facial expression 810 and/or perform voice detection on the recorded video, and then determines that the biological status of the baby is crying. Similarly, a baby sleep as shown in FIG. 8B may be determined by a facial expression and/or an eyes status 820. In an embodiment, the baby transport may perform an instruction in response of the biological status. For example, the system may notify a designated person (e.g., parents) via a voice alert or sending a short message to a remote device when an event is detected according to the biological status. The event may include, but not limited to, hunger, cry, tired, asleep, or discomfort. In some cases, the baby transport may navigate to a designated location or a designated person when a specific event is detected.

In summary, the baby transport provides an autonomous function such that the operation is smoothly executed without using hands, and thus the caregiver could carry other stuff with his/her spare hands. On top of that, the baby transport considers the environment condition and the baby's interest, and thus the safety of the baby is ensured as the nearby obstacle are avoided. Furthermore, giving the baby the view he/she wants brings a comfortable experience to the baby, and thus the baby transport relieves the burden of the caregiver.

Based on the above, several baby transports and methods for operating a baby transport are provided in the present disclosure. The implementations shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A baby transport, comprising:
a car body configured to carry a baby;
a first sensing unit, coupled to the car body, configured to sense a biological signal of the baby;
a second sensing unit, coupled to the car body, configured to sense an environment context; and
a processing unit, coupled to the first sensing unit and the second sensing unit, configured to perform instructions for:
determining a target of interest according to the biological signal of the baby and the environment context; and
planning a route according to the environment context and the target of interest; and
controlling the car body to move according to the route.

2. The baby transport of claim 1, wherein the biological signal of the baby includes an image of the baby, the processing unit is further configured to perform instructions for:
obtaining a head movement, a head pose, a facial expression, a facial feature, a facial gesture, a body pose, an eyeball movement, an eye openness status, an eye blink velocity and amplitude, an eyes gaze vector, a gaze point, a body skeleton and its movement, a gesture, or a combination of the above from the captured image.

3. The baby transport of claim 2, wherein the processing unit is further configured to perform instructions for:
determining a biological status of the baby according to the biological signal of the baby; and
notifying a designated person when an event is detected according to the biological status.

4. The baby transport of claim 3, wherein the event includes hunger, cry, tired, asleep, or discomfort.

5. The baby transport of claim 3, wherein the processing unit is further configured to perform instructions for:
navigating the car body to a designated location when the event is detected.

6. The baby transport of claim 1, wherein the processing unit is further configured to perform instructions for:
monitoring a reaction of the baby when the car body is moving; and
implementing a maneuver in response to the reaction of the baby.

7. The baby transport of claim 6, wherein the maneuver includes stopping, accelerating, decelerating the car body, or making a turn to change a heading of the car body.

8. The baby transport of claim 6, wherein the maneuver includes controlling the car body to tilt down a seat of the car body.

9. A method for operating a baby transport, and the method comprises:
sensing, by a first sensing unit, a biological signal of the baby;
sensing, by a second sensing unit, an environment context;
determining, by a processing unit, a target of interest according to the biological signal of the baby and the environment context;
planning, by the processing unit, a route according to the environment context and the target of interest; and
controlling, by the processing unit, a car body to move according the route.

10. The method of claim 9, wherein the biological signal includes an image of the baby, the processing unit is further configured to perform instructions for:
obtaining a head movement, a head pose, a facial expression, a facial feature, a facial gesture, a body pose, an eyeball movement, an eye openness status, an eye blink velocity and amplitude, an eyes gaze vector, a gaze point, a body skeleton and its movement, a gesture, or a combination of the above from the captured image.

11. The method of claim 10, further comprising:
determining, by the processing unit, a biological status of the baby according to the biological signal of the baby; and
notifying, by the processing unit, a designated person when an event is detected according to the biological status.

12. The method of claim 11, wherein the event includes hunger, cry, tired, asleep, or discomfort.

13. The method of claim 11, further comprising:
navigating, by the processing unit, the car body to a designated location when the event is detected.

14. The method of claim 9, further comprising:
monitoring, by the processing unit, a reaction of the baby when the car body is moving; and
implementing, by the processing unit, a maneuver in response to the reaction of the baby.

15. The method of claim 14, wherein the maneuver includes stopping, accelerating, decelerating the car body, or making a turn to change a heading of the car body.

16. The method of claim 14, wherein the maneuver includes controlling the car body to tilt down a seat of the car body.
